(19) United States
(12) Patent Application Publication
(10) Pub. No.: US 2012/0277844 A1
(43) Pub. Date: Nov. 1, 2012

(54) Biodegradable Drug Eluting stent Pattern

(75) Inventor: Tim Wu, Shrewsbury, MA (US)

(73) Assignee: Tim Wu, Shrewsbury, MA (US)

(21) Appl. No.: 13/476,035

(22) Filed: May 21, 2012

Related U.S. Application Data
(63) Continuation-in-part of application No. 11/843,528, filed on Aug. 22, 2007, now abandoned.
Application No. 11/843,528 is a continuation-in-part of application No. 12/209,104, filed on Sep. 11, 2008, now abandoned.
(60) Provisional application No. 61/488,748, filed on May 22, 2011.
(60) Provisional application No. 60/823,168, filed on Aug. 22, 2006.

(51) Int. Cl. A61F 2/82; A61F 2/84

(52) U.S. Cl. 623/1.11; 623/1.16

(57) ABSTRACT

In embodiment, pattern for polymeric radially expandable implantable medical devices such as stents for implantation into a bodily lumen are disclosed.

### CROSS-REFERENCE TO RELATED APPLICATIONS

This application claims the benefit of the U.S. provisional application No. 61,488,748, filed on May 22, 2011. This application is also a continuation-in-part of the U.S. patent application Ser. No. 11/843,528, filed on Aug. 22, 2007, which claims the benefit of U.S. provisional patent application No. 60/823,168, filed on Aug. 22, 2006. This application is also a continuation-in-part of the U.S. patent application Ser. No. 12/209,104, filed on Sep. 11, 2008, which claims the benefit of U.S. provisional patent application No. 60/578,219, filed on Jun. 8, 2004. This application also claims the benefit of the U.S. provisional application No. 61/368,833, filed on Jul. 29, 2010 and U.S. provisional patent application No. 61/427,141 filed on Dec. 24, 2010. The disclosures of all of which are hereby incorporated by reference in their entireties.

### FIELD OF THE INVENTION

The invention relates to radially expandable polymeric endoprostheses for implantation into luminal structures within the body. In particular, the “endoprostheses” comprises a polymeric structure which polymer is bioabsorbable, biocompatible and structurally configured to fit within luminal structures such as blood vessels in the body. The “endoprostheses” is useful for treating diseases such as atherosclerosis, restenosis and other types of canalicular obstructions.

### BACKGROUND OF THE INVENTION

This invention relates to an endoprostheses for providing mechanical support and a uniform release of drugs to a vessel lumen of a living being.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stem. “Delivery” refers to introducing and transporting the stein through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a retractable sheath or a sock. When the stent is in a desired bodily location, the sheath may be withdrawn which allows the stent to self-expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stem must adequately maintain its size and shape throughout its service life despite the various forces that may come to bear on it, including the cyclic loading induced by the beating heart. For example, a radially directed force may tend to cause a stem to recoil inward. Generally, it is desirable to minimize recoil.

In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stem must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements of a pattern with respect to each other. Thus, a stent pattern may be designed to meet the mechanical requirements of a stent described above which include radial strength, minimal recoil, and flexibility.

Stents have been made of many materials such as metals and polymers, including biodegradable polymer materials. Biodegradable stents are desirable in many treatment applications in which the presence of a stent in a body may be necessary for a limited period of time until its intended function of, for example, maintaining vascular patency and/or drug delivery is accomplished. A stem for drug delivery or a medicated stent may be fabricated by coating the surface of either a metallic or polymeric scaffolding with a polymeric carrier that includes an active agent or drug. An agent or drug may also be mixed or dispersed within the polymeric scaffolding.

In general, there are several important aspects in the mechanical behavior of polymers that affect stent design. Polymers tend to have lower strength than metals on a per unit mass basis. Therefore, polymeric stents typically have less circumferential strength and radial rigidity than metallic stems. Inadequate radial strength potentially contributes to a relatively high incidence of recoil of polymeric stents after implantation into vessels.

Another potential problem with polymeric steals is that their struts or bar arms can crack during crimping and expansion, especially for brittle polymers. The localized portions of the stent pattern subjected to substantial deformation tend to be the most vulnerable to failure. Furthermore, in order to have adequate mechanical strength, polymeric stems may require significantly thicker struts than a metallic stent, which results in an undesirably larger profile.

Another potential problem with polymeric stents is long term creep. Long term creep is typically not an issue with metallic stents. Long term creep refers to the gradual deformation that occurs in a polymeric material subjected to an applied load. Long term creep occurs even when the applied load is constant. Long term creep in a polymeric stent reduces the effectiveness of a stent in maintaining a desired vascular patency. In particular, long term creep allows inward radial forces to permanently deform a stent radially inward.

Therefore, it would be desirable to have polymeric stents with stent patterns that provide adequate radial strength, minimal recoil, and flexibility.

### SUMMARY OF THE INVENTION

The present inventors have proposed novel designs which may employ such bioabsorbable, biocompatible and biodegradable material to make advantageous scaffolds, which may afford a flexibility and stretchability very suitable for implantation in the pulsatile movements, contractions and relaxations of, for example, the cardiovascular system.

Embodiments disclosed herein include, medical devices such as stents, synthetic grafts and catheters, which may or may not comprise a bioabsorbable polymer composition for implantation into a patient.

In one embodiment, a cardiovascular tube-shaped expandable scaffold such as a stent is provided, having a low rejection or immunogenic effect after implantation, which is fabricated from a bioabsorbable polymer composition or blend having a combination of mechanical properties balancing elasticity, rigidity and flexibility, which properties allow bending and crimping of the scaffold tube onto an expandable delivery system for vascular implantation. The instant devices can be used in the treatment of for example, vascular disease such as atherosclerosis and restenosis, and can be provided in a crimpable and/or expandable structure, which can be used in conjunction with balloon angioplasty.

In an embodiment, the medical device can be provided as an expandable scaffold, comprising a plurality of meandering strut elements or structures forming a consistent pattern, such as ring-like structures along the circumference of the device in repeat patterns (e.g., with respect to a stent, without limitation, throughout the structure, at the open ends only, or a combination thereof). The meandering strut structures can be positioned adjacent to one another and/or in oppositional direction allowing them to expand radially and uniformly throughout the length of the expandable scaffold along a longitudinal axis of the device. In one embodiment, the expandable scaffold can comprise specific patterns such as a lattice structure, beecomb structure or dual-helix structures with uniform scaffolding with optionally side branching.

In one embodiment, a bioabsorbable and flexible scaffold circumferential about a longitudinal axis so as to form a tube, the tube having a proximal open end and a distal open end, and being expandable from an unexpanded structure to an expanded form, and being crimpable, the scaffold having a patterned shape in expanded form comprising: a) the first plurality of pairs of radially expandable undulating cylindrical rings that are longitudinally aligned and are connected at a plurality of intersections by S-shaped links to form a plurality of beecomb cells. Each adjacent S-shaped links were sited in an opposite direction to provide adequate free space for the second plurality of ring to cross. And b) a plurality of second radially expandable undulating cylindrical rings that are shorter than the first radially expandable undulating cylindrical rings and longitudinally aligned across the middle of each beecomb cells to form circumferentially X-shaped patterns. The meandering between beecomb cell structure and X-shaped undulations along the longitudinal axis form a unique pattern that provides the device both the flexibility and radial strength once it being expanded.

In one embodiment, both the first and second plurality of radially expandable undulating cylindrical rings are essentially sinusoidal. In another embodiment, each of the second strut patterns can be found at the proximal open end and the distal open end. In one embodiment, each of the second strut patterns is further found between the proximal open end and the distal open end.

In one embodiment, the intersection links among the first plurality of radially expandable undulating cylindrical rings can be S-shaped, straight line or non sinusoidal curves. In another embodiment, the two pluralities of radially expandable undulating cylindrical rings can be linked at one point, two points, or any other multiple points and the link sites can be between two peaks (peak-peak), peak-valley and middle-middle of stent's strut.

In another embodiment, the scaffold comprises a structure wherein the two first undulating cylindrical rings were linked on each peaks by S-shaped structure with opposite direction to provide maximum space on each side of the S-shaped structure for the second undulating cylindrical ring's easy crossing. In one embodiment, the intervening between the second undulating cylindrical rings and each S-shaped linking structure form a unique meandering strut pattern to provide more radial strength of the invented scaffold.

In one embodiment, the scaffold can comprise a structure wherein each of the second strut patterns can be found between the proximal open end and the distal open end but not at the proximal open end or distal open end. In another embodiment, the scaffold can comprise a structure wherein the second strut patterns can be found at least one of the proximal open end or the distal open end.

In a specific embodiment, the scaffold comprises a stem having an unexpanded configuration and an expanded configuration; an outer tubular surface and an inner tubular surface, the stent comprising: a plurality of biodegradable, paired, separate circumferential bands having a pattern of distinct undulations in an unexpanded configuration and substantially no undulations in an expanded configuration, the undulations of the biodegradable, paired, separate circumferential bands in the stent in an unexpanded state being incorporated into a substantially planar ring in an expanded state, and a plurality of biodegradable interconnection structures spanning between each pair of circumferential bands and connected to multiple points on each band of the paired bands.

In an embodiment, the stent interconnecting structures comprise a pattern of undulations both in alt unexpanded and expanded configuration. In an alternate embodiment, the interconnection structures comprise a pattern containing no undulations in both an unexpanded and expanded configuration. The interconnection structures of the stent can expand between undulations of paired circumferential bands.

In one embodiment, at least one of the plurality of paired biodegradable circumferential bands includes along its outer tubular surface, a radio-opaque material capable of being detectable by radiography, MRI or spiral CT technology. Alternatively, at least one of the interconnection structures includes a radio-opaque material along its outer tubular surface, which can be detectable by radiography, MRI or spiral CT technology. The radio-opaque material can be housed in a recess on one of the circumferential bands, or in a recess on one of the interconnection structures. In one embodiment, at least one of the interconnection structures and at least one of the circumferential bands includes a radio-opaque material along the outer tubular surface, which is detectable by radiography, MRI or spiral CT technology.

In an alternate embodiment, a method for fabricating a tube-shaped scaffold comprising: preparing a racemic poly-lactide mixture; fabricating a biodegradable polymer tube of the racemic poly-lactide mixture; laser cutting the tube until such scaffold is formed. In another embodiment, the fabrication of the scaffold can be performed using a molding technique, which is substantially solvent-free, or an extrusion technique.

There is also provided a method for fabricating the tube-shaped scaffold comprising, blending a polymer composition comprising a crystallizable composition comprising a base polymer of poly L-lactide or poly D-lactide and Amorphous Calcium Phosphate(ACP) nanoparticle, molding the polymer composition to structurally configure the scaffold; and cutting the scaffold to form the desired scaffold patterns. In this embodiment, the blended composition comprises a racemic mixture of poly L-lactide and poly-D lactide. Accordingly, medical devices such as a stent, produced by this method consist essentially of a racemic mixture of a poly-L and poly-D lactide. In this embodiment, the stent can comprise other polymer materials such as trimethylcarbonate. In embodiment wherein the device comprises trimethylcarbonate, the amount of trimethylcarbonate does not exceed more than 40% of the weight of the stent.

The tube-shaped scaffold can also comprise one or more than one pharmaceutical substances, which can be encapsulated within the polymeric structure for release of the drugs locally and for the treatment and prevention of tissue inflammation and platelet aggregation. The tube-shaped scaffold can also comprise at least one attached or embedded identification marker, which can be attached or embedded identification marker comprising a spot radioopacity or a diffuse radioopacity.

The tube-shaped scaffold can also comprise meandering struts which can be interlocked by means of ringlet connectors comprising configurations selected from one or more of the groups consisting of shaped-like an H, shaped-like an X, perforated circle, double adjacent H, triple adherent connection, two adjacent parallel connections, sinusoidal connect of parallel struts.

In another embodiment, a bioabsorbable and flexible scaffold circumferential about a longitudinal axis so as to form a tube, the tube having a proximal open end and a distal open end, and being crimpable and expandable, and having a patterned shape in expanded form comprising, a first multicomponent strut pattern helically coursing from the proximal open end to the distal open end of the tube; a second multicomponent strut pattern helically coursing from the proximal open end to the distal open end of the tube; wherein a component of the first multicomponent strut pattern opposes by from about 120.degree. to about 180.degree. a component of the second multicomponent strut pattern as each helically courses from the proximal open end to the distal open end of the tube. In one embodiment, the scaffold comprises a structure wherein each component strut pattern of the first multicomponent strut pattern is substantially the same in configuration. The scaffold can also comprise a structure wherein each component strut pattern of the second multicomponent strut pattern is substantially the same in configuration. Alternatively, the scaffold can comprise a structure wherein each component strut pattern of the first and second multicomponent strut pattern is substantially the same in configuration. In this embodiment, that is, wherein each opposing component of the component strut pattern between the first multicomponent strut pattern and second multicomponent strut pattern is substantially the same in configuration.

The scaffold can comprise a third multicomponent strut pattern helically coursing from the proximal open end to the distal open end of the tube. The scaffold can further comprise a fourth multicomponent strut pattern helically coursing from the proximal open end to the distal open end of the tube, and a fifth multicomponent strut pattern helically coursing from the proximal open end to the distal open end of the tube. Each helix of a pair of the multicomponent strut patterns may turn about the tube in a left-handed screw direction. Alternatively, the scaffold can comprise a structure wherein each helix of both of the multicomponent strut patterns turns about the tube in a right-handed screw direction. In a further embodiment, at least one helix of both of the multicomponent strut patterns turns about the tube in a left-handed screw direction while another helix turns in a right-handed screw direction. In yet another embodiment, all of the helices of the multicomponent strut patterns turns about the tube in the same-handed direction.

There is also provided, a flexible scaffold circumferential about a longitudinal axis so as to form a tube, the tube having a proximal open end and a distal open end, and being crimpable and expandable, and having a patterned shape in unexpanded form comprising; a first sinusoidal strut pattern comprising a series of repeated sinusoids defined by an apex section and a trough section, the repeated sinusoids coursing from the proximal open end to the distal open end of the tube; and a second sinusoidal strut pattern comprising a series of repeated sinusoids defined by an apex section and a trough section, the sinusoids of the second sinusoidal strut pattern being double or triple of first sinusoid strut pattern.

In one embodiment, the scaffold can comprise a structure wherein the first sinusoidal strut pattern and the second sinusoidal strut pattern are repeated multiple times, one after the other to form the scaffold; or wherein the first sinusoidal strut pattern and the second sinusoidal strut pattern are the same; or wherein the first sinusoidal strut pattern and the second sinusoidal strut pattern are different. The scaffold can be made of a biodegradable material, such as poly-lactide.

In another embodiment, a bioabsorbable and flexible scaffold circumferential about a longitudinal axis so as to for a tube, the tube having a proximal open end and a distal open end, and being crimpable and expandable, and having a patterned shape in unexpanded form comprising; a first sinusoidal strut pattern comprising a series of repeated sinusoids defined by an apex section and a trough section, the repeated sinusoids coursing from the proximal open end to the distal open end of the tube; a second sinusoidal strut pattern comprising a series of repeated sinusoids defined by an apex section and a trough section, the sinusoids of the second sinusoidal strut pattern being in phase with respect to the apex and the troughs of the first sinusoidal strut pattern; wherein the second sinusoidal strut pattern is connected to the first sinusoidal strut pattern at at least two points, and wherein the connection at the points is from an apex of a sinusoid of the first sinusoidal pattern to an apex of a sinusoid of the second sinusoidal pattern.

In this embodiment, the first sinusoidal strut pattern and the second sinusoidal strut pattern are repeated multiple times, one after the other form the scaffold; the first sinusoidal strut pattern and the second sinusoidal strut pattern are the same or different. The scaffold is made of a biodegradable material, such as a polymer such as a poly-lactide polymer; and comprises a structure wherein the second sinusoidal strut pattern is connected to the first sinusoidal strut pattern at at least three or four points.

In an embodiment wherein the tubular-shaped structure is a stent, the stent comprises a plurality of sinusoidal-like or meandering strut patterns encompassing the diameter of the tubular structure, wherein each sinusoidal ring-like structure can be continuous with an adjacent sinusoidal ring-like structure at a point. Adjacent sinusoidal/meandering patterns can be continuous at at least one point. In one embodiment, the stem scaffold can be formed by two different types of meandering elements, the first meandering element comprises a zig-zag pattern/sinusoidal-like structure comprising with peaks and valleys which can extend the entire circumference of the scaffold, so that the meandering element can maintain a sinusoidal shape even when the scaffold structure is in its fully expanded configuration. A second type of meandering element also forms the stent scaffold, and can be intercalated or positioned in between adjacent first meandering elements, so that when the scaffold structure is fully deployed, the second type of meandering element forms a ring-like or hoop structure which can adapt to fully fit the diameter of a tubular organ space where the scaffold is deployed. The ring-like (also referred to as ringlet) element provides the tubular scaffold with increased hoop strength and can prevent collapsing of the scaffold once deployed. More specifically, this embodiment provides the ring, or hoop its expanded state, at least at one end of the tubular device for securing or anchoring the scaffold position in the organ space. The embodiment can also provide a plurality of ringlets distributed randomly or in a regularly spaced pattern along the length of the scaffold. In the case of an expanded scaffold, the ringlets are designed to expand utmost into a ring or hoop shape or expand to a degree so as to retain some sinusoidal shape for more flexible, less rigid structural characteristics. The presence of secondary meandering struts both in the hoop shape at a scaffold end or anywhere along the scaffold axis, aids in preventing scaffold “creep” by tightly pushing against the wall of the organ space, as e.g. cardiovascularity. “Creep” in the present invention is defined as gradual dislocation of an implant from the original emplacement in the organ space.

This change as caused by pulsating organ walls as well as bodily fluid flux, can be countered by re-crystallized hoop or ring entities that span the luminal space, press tightly against the surrounding tissue and yet exhibit enough elasticity and compatibility to reduce local injurious impact.

In one embodiment, the tubular scaffold can comprise one or more than one of a second type of meandering elements and can be positioned in the tubular scaffold at alternating patterns between a first type of meandering elements to form a repeat pattern depending of the desired length of the tubular scaffold. In another embodiment, there is provided a scaffold configuration comprising meandering strut elements connected to an expansion-stabilizing ring-shaped portion.

A medical device embodiment, such as a stent, may be manufactured from polymeric materials which comprises a polymer having breakdown moieties that are “friendly” at contact with bodily tissues and fluids such as the vascular wall. In a specific embodiment, the medical device comprises a polymer with breakdown kinetics sufficiently slow to avoid tissue overload or inflammatory reactions which can lead to restenosis, for example, which provides a minimum of 30-day retention of clinically supportive strength. In one embodiment the medical device may be endured in place as much as 3-4 months post-implantation without undergoing substantial bioabsorption.

In one embodiment, the implant can undergo transitional change after implantation, from a solid flexible implant at implantation, to a “rubbery state” post-implantation which exhibits flexibility, yet enough resilience and cohesion so as to permit surgical intervention.

In one embodiment, the polymer selected for making the device has flexibility and elasticity suitable for an implant in friction-free contact with vascular walls during the cardiovascular pulsing contractions and relaxations. In an embodiment, the medical device comprises a stretchable and elastic scaffold, which has a sufficiently rigid strength to be capable of withstanding the fluctuating cardiovascular pressures within a blood vessel. For example, the polymer selection can be based on evaluation criteria based on mass loss in terms of decreased molecular weight, retention of mechanical properties, and tissue reaction.

In an embodiment, the polymer composition allows polymer realignment and the development of a crystalline morphology. Plastic deformation imparts crystallinity to polymer molecules. A polymer in crystalline state is stronger than its amorphous counterpart. In stent embodiments comprising ring-like structures, the ring-like structures or ringlet may be a material state that is inherently stronger than that of a sinusoidal stent segment. that can enhance the mechanical properties of the medical device, enhance processing conditions, and provide potential of cross-moiety crystallization, for example, thermal cross-links.

In another device embodiment, the medical device comprises a polymer blend comprising a marker molecule, for example, radio-opaque substance, a fluorescent substance or a luminescent substance, which can serve to detect or identify the medical device once implanted into a patient. For example, compounds that can be used as marker molecules include, iodine, phosphorous, fluorophores, and the like. A medical device such as one employing fluoroscopy, X-rays, MRI, CT technology and the like may be used to detect the radioopaque substance.

In this and other embodiments of the invention, the medical device can comprise fillers and one or more pharmaceutical substances for local delivery. The medical device may, for example, comprise, a biological agent, a pharmaceutical agent, e.g. an encapsulated drug (which may be used for localized delivery and treatment—for example, of vascular wall tissue and lumen).

In another embodiment, there is provided a scaffold structure comprising a core degradation schedule which provides more specifically a simultaneously slow release of medication for the treatment and prevention of tissue inflammation and platelet aggregation. The polymer composition or blend provides uniform degradation in situ avoiding polymer release in large chunks or particles.

In another embodiment, the polymer compositions are used to manufacture medical device for implantation into a patient. The medical devices comprise scaffolds having biodegradable, bioabsorbable and nontoxic properties and include, but are not limited to stents, stent grafts, vascular synthetic grafts, catheters, vascular shunts, valves and the like. Biocompatible and bioabsorbable scaffolds may be particularly found useful in treatment of coronary arteries. For example, a scaffold structure may be manufactured or extruded from a composition comprising a base polymer material, at least one drug for local delivery and at least one attached or embedded identification marker.

In another embodiment, a method for treating vascular disease is disclosed, the method comprising, administering to a person suffering with vascular disease a medical scaffold or device comprising a structure made from a biocompatible, bioabsorbable polymer.

### BRIEF DESCRIPTION OF THE DRAWINGS

The figures provided herewith depict embodiments that are described as illustrative examples that are not deemed in any way as limiting the present invention.

The patent or application file contains at least one drawing executed in color. Copies of this patent or patent application publication with color drawing(s) will be provided by the Office upon request and payment of the necessary fee.

FIG. 1A is two dimensional Auto CAD drawing depicting a fully view of an embodiment of a bioabsorbable medical device depicting a scaffold strut segments, nested hoop structures, end ring, meandering and marker pocket regions.

FIG. 1B is a computer simulation illustration depicting a partial view of a bioabsorbable medical device depicting a scaffold strut segments, nested hoop structures, end ring, meandering and marker pocket regions.

FIG. 1C is a photo image of a bioabsorbable medical device in an expanded configuration showing that the nested hoop or ring structure, end ring and meandering strut pattern.

FIG. 2A is a computer simulation illustration depicting a partial view of a bioabsorbable medical device depicting the first plurality of pairs of radially expandable undulating cylindrical rings.

FIG. 2B is a two dimensional Auto CAD drawing depicting a partial view of an embodiment of a bioabsorbable medical device depicting the first plurality of pairs of radially expandable undulating cylindrical rings that are longitudinally aligned and are connected at a plurality of intersections by S-shaped links to form a plurality of beecomb cells.

FIG. 2C is computer simulation illustration depicting a partial view of a bioabsorbable medical device depicting the first plurality of pairs of radially expandable undulating cylindrical rings that are longitudinally aligned and are connected at a plurality of intersections by S-shaped links to form a plurality of beecomb cells.

FIG. 3A is a two dimensional Auto CAD drawing showing a partial view of a bioabsorbable medical device depicting a plurality of second radially expandable undulating cylindrical rings that are shorter than the first radially expandable undulating cylindrical rings and longitudinally aligned across the middle of each beecomb cells to form circumferentially a X-shaped patterns.

FIG. 3B is computer simulation illustration depicting a partial view of a bioabsorbable medical device depicting a plurality of second radially expandable undulating cylindrical rings that are shorter than the first radially expandable undulating cylindrical rings and longitudinally aligned across the middle of each beecomb cells to form circumferentially X-shaped patterns.

FIG. 4A is a two dimensional Auto CAD drawing showing a partial view of a bioabsorbable medical device depicting the meandering between the first plurality of pairs of radially expandable undulating cylindrical rings and a second plurality of radially expandable undulating cylindrical rings that are shorter than the first radially expandable undulating cylindrical rings

FIG. 4B is computer simulation illustration depicting a partial view of a bioabsorbable medical device depicting the meandering between the first plurality of pairs of radially expandable undulating cylindrical rings and a second plurality of radially expandable undulating cylindrical rings that are shorter than the first radially expandable undulating cylindrical rings

FIG. 5 is a two dimensional Auto CAD drawing showing a planar view of an alternate embodiment of a bioabsorbable stent scaffold structure showing alternate design for the strut elements in expanded configuration and hoop/ring elements.

FIG. 6A: depict a partial view of an alternate embodiment of a bioabsorbable stent scaffold structure showing alternate design for the strut elements in expanded configuration, end hoop, radial opaque marker pocket elements.

FIG. 6B: depict a partial view of an alternate embodiment of a bioabsorbable stent scaffold structure showing alternate design for the strut elements in expanded configuration, end hoop, radial opaque marker pocket elements.

FIG. 7: depicts the bioabsorbable stent crimped on an expandable balloon catheter.

FIG. 8: depict the bioabsorbable stem of FIG. 7 in an expanded condition.

FIG. 9: is an x-ray image depicting a biodegradable stent expanded in pig coronary artery.

FIG. 10: are pathological images depicting the invented biodegradable stent in pig coronary artery at one month post implantation.

### DETAILED DESCRIPTION

Disclosed herein are novel structure elements, and novel compositions which may be used to make such novel structural elements. The present embodiments may find use in the treatment of many diseases and physiological ailments.

In recent years, metallic stents have come into use to aid in the clearing the clogged lumen of the vascular system. However, the efficacy of metallic stent implants in vascular arteries has been diminished by certain disadvantageous results. For example, since such stents have shown a tendency to stimulate formation of scar tissue or restenosis in the wound inflicted in the vascular area of deployment. This effect becomes more detrimental in the use of small diameter tubes in therapy. Moreover, it is important to avoid arterial wall damage during stent insertion. These factors (although somewhat difficult to control in the first instance) are aimed at trying to reduce the mechanical reasons that lead to excessive clot and scar formation within the vessel lumen.

Stent structures typically comprise a number of meandering patterns. By “meandering” it is meant moving along a path that is other than strictly linear. Due to the need to have an unexpanded form to allow for easy insertion of a stent into its biological milieu, such as, without limitation, the vasculature, the meandering patterns making up a stent are often sinusoidal in nature, that is having a repeating sequence of peaks and troughs. Often such sinusoidal structures are normalized such that each peak or trough is generally of the same distance as measured from a median line. By “non-sinusoidal” it is meant a pattern not having a repeating sequence of peaks and valleys, and not having a series of raised portions of generally the same distance as measured from a median line nor a series of depressed portions of generally the same distance as measured from a median line. A stent may be characterized as having three distinct configurations, an unexpanded state (as manufactured), a crimped state (a compressed state as compared to the unexpanded state), and an expanded state (as deployed as an implant in vivo).

While the configurations disclosed herein are not limited to fabrication by any particular material, in certain embodiments such configurations are constructed from a flexible, elastic, and bioabsorbable plastic scaffold. In embodiments disclosed herein, there is illustrated a bioabsorbable and expandable scaffold of various shapes, patterns, and details fabricated from bioabsorbable polymers and polymer compositions. The scaffolds in an advantageous embodiment balance the properties of elasticity; rigidity and flexibility while being more biocompatible, less thrombogenic and immunogenic than prior art polymeric medical devices. Such embodiments may provide means for preventing device creep or repositioning when crimpedly placed on a carrier as well as when expandedly placed in a living organ space. Stent implants may employ a balloon expandable medical device which comprises a thermal balloon or non-thermal balloon.

For the purposes of the present invention, the following terms and definitions apply:

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

Furthermore, a property of a material that quantifies a degree of strain with applied stress is the modulus. “Modulus” may be defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, and the strain rate or rate of deformation. For example, below its T.sub.g, a polymer tends to be brittle with a high modulus. As the temperature of a polymer is increased from below to above its T.sub.g, its modulus decreases.

The “ultimate strength” or “strength” of a material refers to the maximum stress that a material will withstand prior to fracture. A material may have both a tensile and a compressive strength. The ultimate strength may be calculated from the maximum load applied during a test divided by the original cross-sectional area.

The term “elastic deformation” refers to deformation of an object in which the applied stress is small enough so that the object moves towards its original dimensions or essentially its original dimensions once the stress is released. However, an elastically deformed polymer material may be prevented from returning to an undeformed state if the material is below the T.sub.g of the polymer. Below T.sub.g, energy barriers may inhibit or prevent molecular movement that allows deformation or bulk relaxation.

“Elastic limit” refers to the maximum stress that a material will withstand without permanent deformation. The “yield point” is the stress at the elastic limit and the “ultimate strain” is the strain at the elastic limit. The term “plastic deformation” refers to permanent deformation that occurs in a material under stress after elastic limits have been exceeded.

Various embodiments of stent patterns for polymeric stents are disclosed herein. Stents may be composed partially or completely of polymers. In general, polymers can be biostable, bioabsorbable, biodegradable, or bioerodible. Biostable refers to polymers that are not biodegradable. The terms biodegradable, bioabsorbable, and bioerodible, as well as degraded, eroded, and absorbed, are used interchangeably and refer to polymers that are capable of being completely eroded or absorbed when exposed to bodily fluids such as blood and can be gradually resorbed, absorbed and/or eliminated by the body.

A stent made from a biodegradable polymer is intended to remain in the body for a duration of time until its intended function of, for example, maintaining vascular patency and/or drug delivery is accomplished. After the process of degradation, erosion, absorption, and/or resorption has been completed, no portion of the biodegradable stent, or a biodegradable portion of the stent will remain. In some embodiments, very negligible traces or residue may be left behind. The duration can be in a range from about a month to a few years. However, the duration is typically in a range from about six to twelve months.

The general structure and use of stents will be discussed first in order to lay a foundation for the embodiments of stent patterns herein. In general, stents can have virtually any structural pattern that is compatible with a bodily lumen in which it is implanted. Typically, a stent is composed of a pattern or network of circumferential rings and longitudinally extending interconnecting structural elements of struts or bar arms. In general, the struts are arranged in patterns, which are designed to contact the lumen walls of a vessel and to maintain vascular patency. A myriad of strut patterns are known in the art for achieving particular design goals. A few of the more important design characteristics of stents are radial or hoop strength, expansion ratio or coverage area, and longitudinal flexibility.

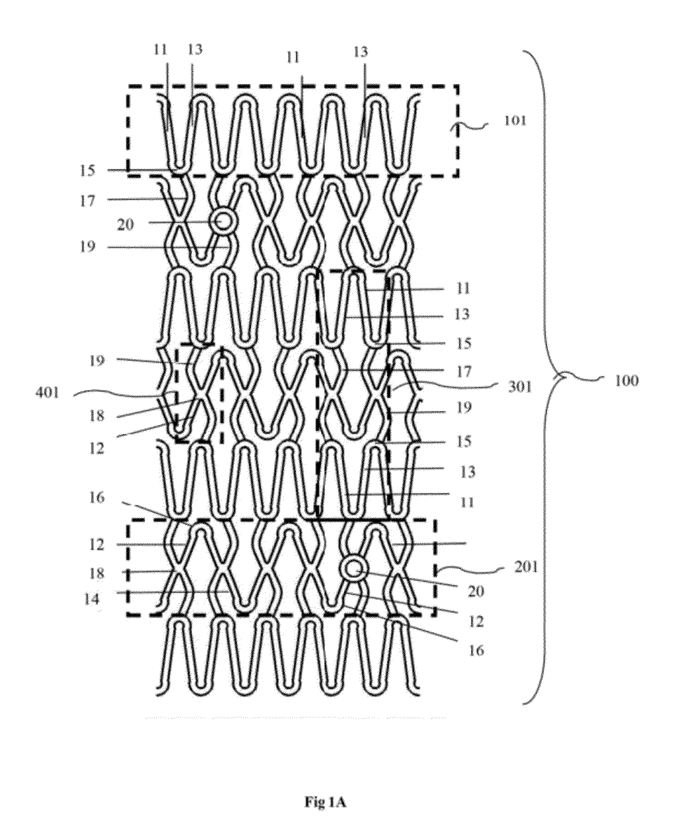

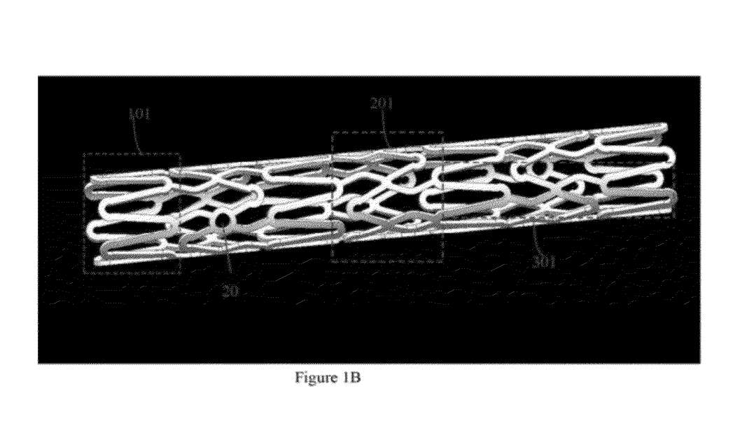

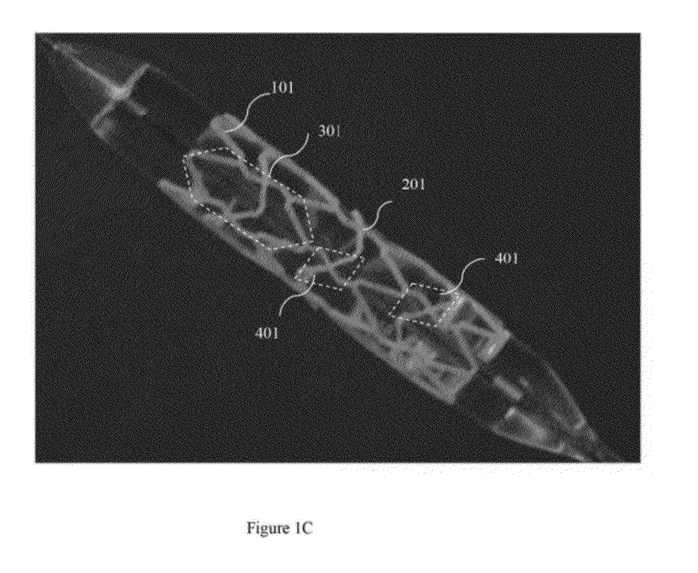

Now turning to the figures, FIG. 1A is two dimensional Auto CAD drawing depicting a fully view of an embodiment of a bioabsorbable stent 100 depicting: 1) the first plurality of pairs of radially expandable undulating cylindrical rings 101, 2) the second plurality of radially expandable undulating cylindrical rings 201 that are shorter than the first radially expandable undulating cylindrical rings, and 3) the meandering between the first plurality of pairs of radially expandable undulating cylindrical rings and the second plurality of radially expandable undulating cylindrical rings to form a sinusoidal structure 301. The repeating of meandering structure 301 further forms a tubular scaffolding structure of stent.

FIG. 1B is a computer simulation illustration depicting a partial view of a bioabsorbable medical device in three dimension depicting the first plurality of pairs of radially expandable undulating cylindrical rings 101, the S-shaped connection 17 and 19 between the first plurality of pairs of radially expandable undulating cylindrical rings 101, the second plurality of radially expandable undulating cylindrical rings 201 that are shorter than the first radially expandable undulating cylindrical rings, and 3) the meandering between the first plurality of pairs of radially expandable undulating cylindrical rings 101 and the second plurality of radially expandable undulating cylindrical rings 201 to form a sinusoidal structure 301 of stent.

FIG. 1C is a photograph of a bioabsorbable stent scaffold embodiment as manufactured from FIG. 1A design showing a bioabsorbable medical device in a expanded configuration showing the first plurality of pairs of radially expandable undulating cylindrical rings 101, the second plurality of radially expandable undulating cylindrical rings 201, the meandering between the first plurality of pairs of radially expandable undulating cylindrical rings 101 and the second plurality of radially expandable undulating cylindrical rings 201 to form a sinusoidal structure 301, and 4) the x-shaped structures 401 formed during the second plurality of radially expandable undulating cylindrical rings cross each S-shaped links between the first plurality of radially expandable undulating cylindrical rings.

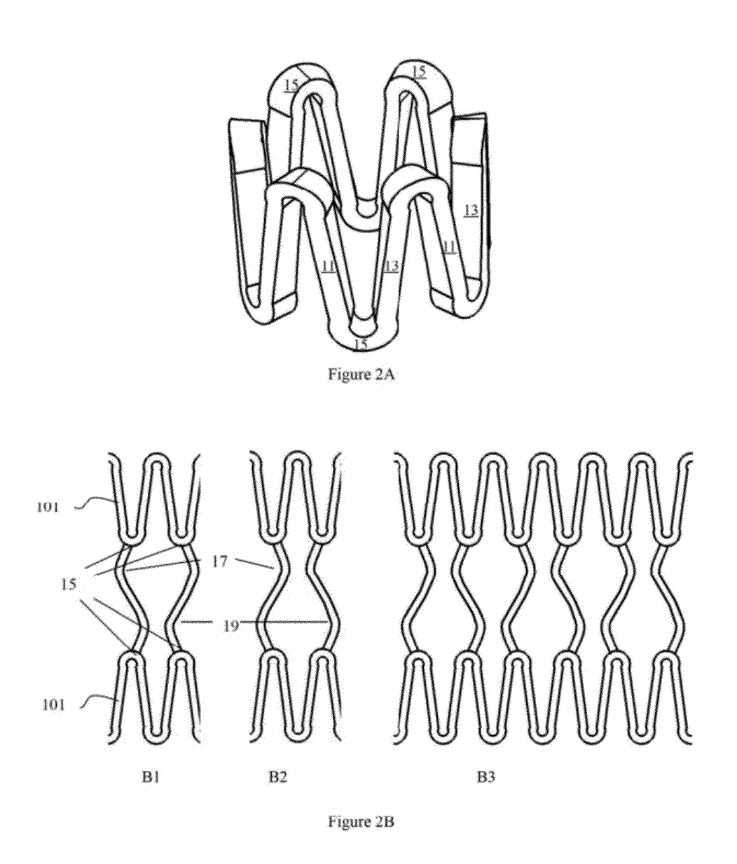

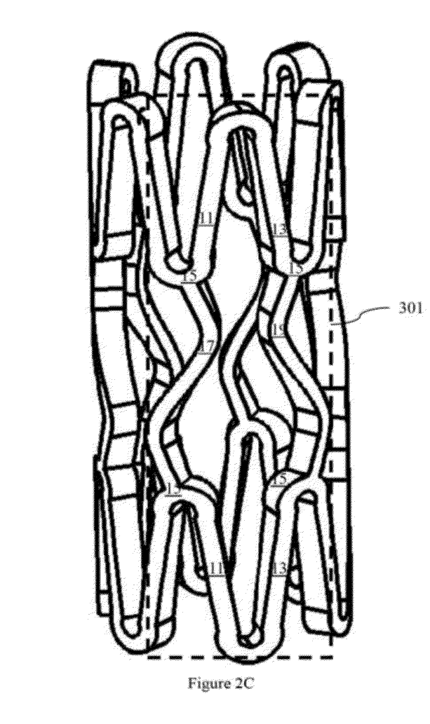

FIG. 2A is a computer simulation illustration depicting a partial view of a bioabsorbable medical device depicting the first plurality of pairs of radially expandable undulating cylindrical rings 101. As showed in the drawing, the ring is sinusoidal structure with multiple peaks 15 and V-shaped waving-arms of 11 and 13.

FIG. 2B is a two dimensional Auto CAD drawing depicting a partial view of an embodiment of a bioabsorbable medical device depicting the first plurality of pairs of radially expandable undulating cylindrical rings 101 that are longitudinally aligned and are connected at a plurality of point of 15 by S-shaped links 17 and 19 to form a plurality of beecomb cells 301.

FIG. 2C is a three dimensional illustration with computer simulation depicting a partial view of a prematurely expanded bioabsorbable medical device depicting the first plurality of pairs of radially expandable undulating cylindrical rings 101 that are longitudinally aligned and are connected at a plurality point 15 by S-shaped links 17 and 19 to form a plurality of beecomb cells 301.

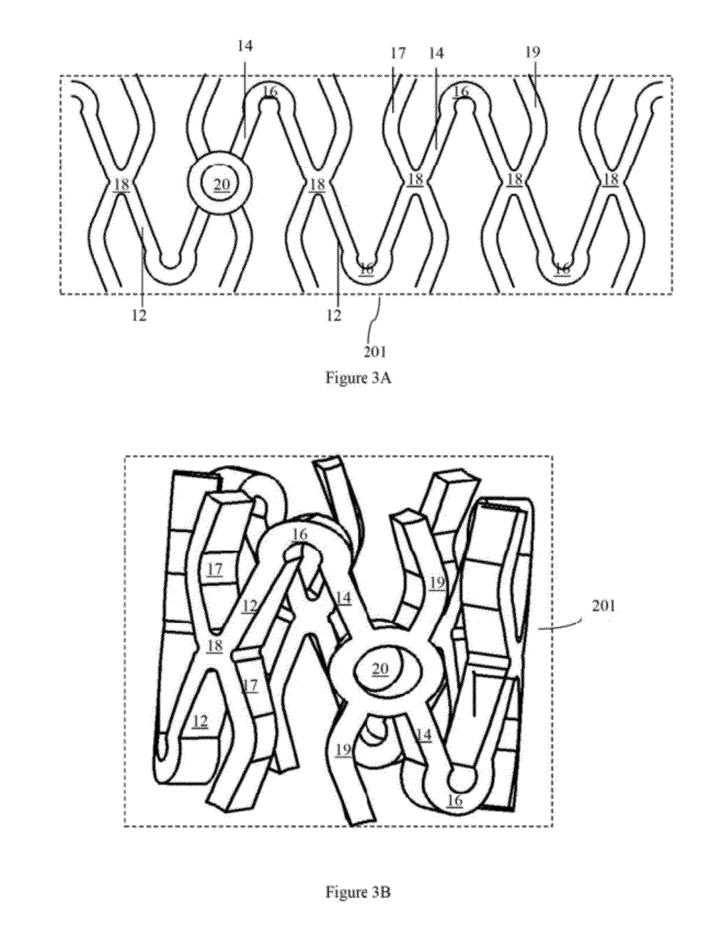

FIG. 3A is a two dimensional Auto CAD drawing showing a partial view of a bioabsorbable medical device depicting a plurality of second radially expandable undulating cylindrical rings 201 that composed with multiple peaks point 16 and V-shaped waving arms 12 and 14. The second radially expandable undulating cylindrical rings are shorter than the first radially expandable undulating cylindrical rings 101 and longitudinally aligned across the middle point 18 in each beecomb cells 301 to form circumferentially multiple X-shaped patterns 401 and the pocket 20 in the crossing area for radiopaque material.

FIG. 3B is a three dimensional illustration with computer simulation depicting a partial view of a bioabsorbable medical device depicting a plurality of second radially expandable undulating cylindrical rings 201, which are longitudinally aligned across the middle of each beecomb cells 301 to form circumferentially a series of X-shaped patterns 401 and the pocket 20 in the crossing area for radiopaque material.

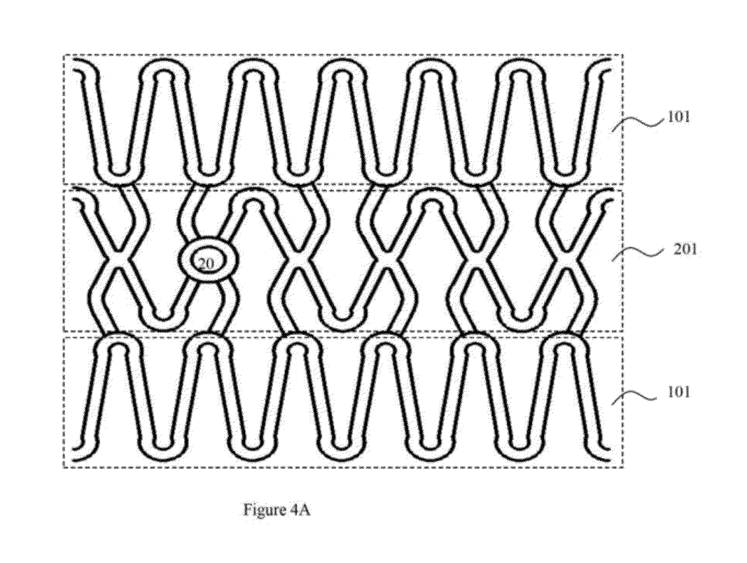

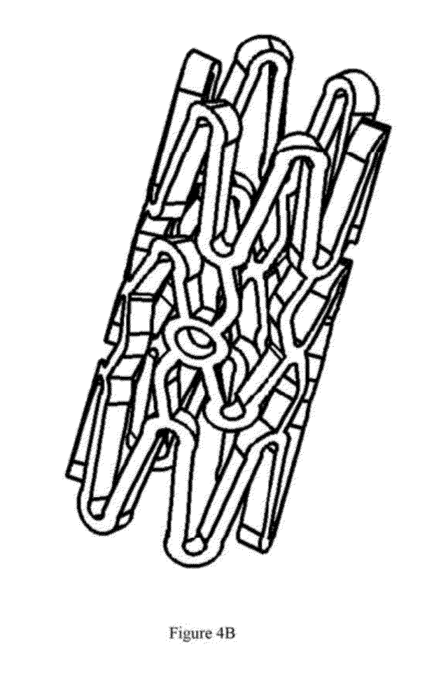

FIG. 4A is a two dimensional Auto CAD drawing showing a partial view of a bioabsorbable medical device depicting the meandering between the first plurality of pairs of radially expandable undulating cylindrical rings 101 and a second plurality of radially expandable undulating cylindrical rings 201 that are shorter than the first radially expandable undulating cylindrical rings and the pocket 20 in the crossing area for radiopaque material.

FIG. 4B is the further illustration of the meandering structure with a computer simulation depicting a partial view of a bioabsorbable medical device depicting the meandering structure between the first plurality of pairs of radially expandable undulating cylindrical rings and a second plurality of radially expandable undulating cylindrical rings.

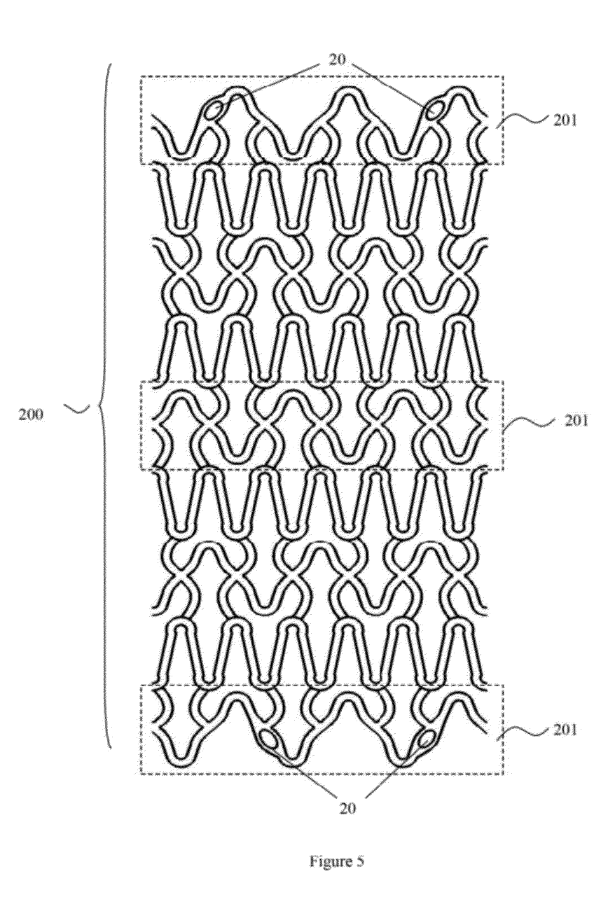

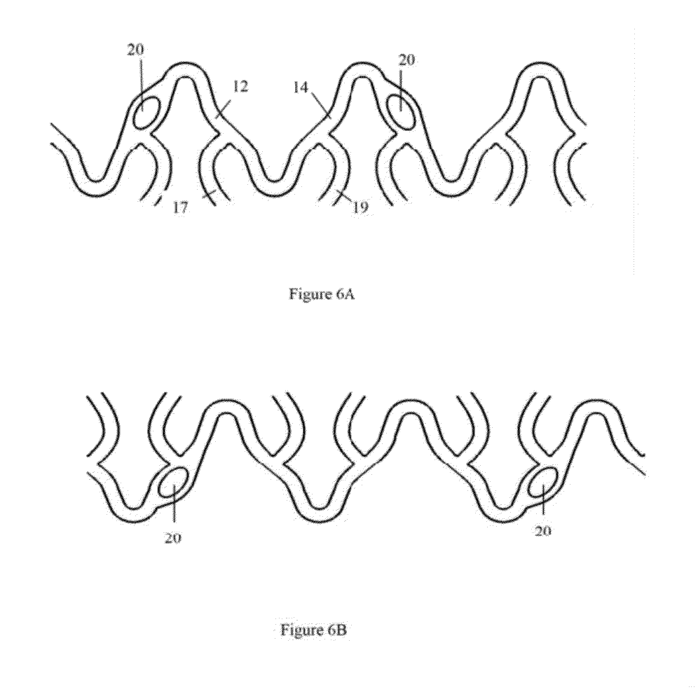

FIGS. 5, 6A and 6B are two dimensional Auto CAD drawing depicting the planar view of an alternate embodiment of a bioabsorbable stent scaffold structure showing alternate design for the strut elements in expanded configuration, end hoop, radial opaque marker pocket elements 20.

In general, a stent pattern is designed so that the stent can be radially expanded (to allow deployment). The stresses involved during expansion from a low profile to an expanded profile are generally distributed throughout various structural elements of the stent pattern. As a stent expands, various portions of the stent can deform to accomplish a radial expansion.

In one embodiment, the invented biodegradable stent has increased radial strength and geometric stability. FIGS. 2A, 2B and 2C depicts one embodiment of a stent 100 pattern. In FIG. 2B, a portion of a stent pattern 301 is shown in a flattened condition so that the pattern can be clearly viewed. When the flattened portion of stem pattern 301 is in a cylindrical condition, it forms a radially expandable stent FIG. 2C. The stent is typically formed from a tubular member, but it can be formed from a flat sheet such as the portion shown in FIG. 2B and rolled and bonded into a cylindrical configuration.

FIG. 2B (B1 and B2) depicts two pairs beecomb-shaped cell 301 with S-shaped links 17 and 19 in opposite direction. Pairs 301 form more free space at each direction for ring 201 to cross from the center of each link to form multiple X-shaped patterns 401. As these X-shaped patterns 401 will transit to +-shaped structure with stent expansion as showed in FIG. 1C, the radial strength in each beecomb-shaped cells will be reinforced. Embodiments of stent 100 may have any number of pairs 301. Each pair 301 was then connected in an opposite direction to form a multiple beecomb-shaped cells circumferentially and longitudinally.

As depicted in FIG. 2C, each pair beecomb-shaped cell 301 consist two rings 101 connected with S-shaped links 17 and 19 in opposite direction and are longitudinally aligned and are connected at a plurality of intersections to form a plurality of beecomb-shaped cells 301. Beecomb-shaped cells 310 may be described in part as having two adjacent S-shaped regions 17 and 19 in an opposite direction and two V-shaped undulating rings 11 and 13. Embodiments of the stent depicted in FIG. 2B can include any number of beecomb-shaped regions or cells along a circumferential direction and rings along the longitudinal axis. It is a known art that the beecomb-shaped cells enhance the geometric stability of the stein.

Some embodiments of the stent in FIGS. 2B and 2C may include holes or depots 20 to accommodate radiopaque material. The stent may be visualized during delivery and deployment using X-Ray imaging if it contains radiopaque materials. By looking at the position of stent with respect to the treatment region, the stent may be advanced with the catheter to a location. In one embodiment, depots or holes may be drilled using a laser.

In one embodiment, the biodegradable stent have varied stiffness and flexibility once expanded inside the artery. FIGS. 3A and 3B depict a partial view of a bioabsorbable medical device depicting a plurality of second radially expandable undulating cylindrical rings 201 that are shorter than the first radially expandable undulating cylindrical rings 101 and longitudinally aligned across the middle of each beecomb cells to form circumferentially multiple X-shaped patterns and further transit to +-shaped structure with the stent, expansion to structurally reinforce the radial strength of each beecomb cells.

The stiffness or flexibility of a portion of a stent pattern can depend on the mass of the portion of the stent. The mass of a portion may be varied by varying the width and/or length of strut or bar arm that makes up a portion. The shorter a strut, the stiffer and less flexible it is. The smaller the width of a stein, the less stiff and more flexible it is. In addition, a portion with a smaller mass may tend to undergo more deformation. By allocating the amount of mass to specific struts, it is possible to create a stent having variable strength with greater strength at the high mass areas.

In addition, deformation of portions of a stent during radial expansion can also influence a stent's radial strength, recoil, and flexibility. In general, deformation of a polymeric material may induce alignment or increase the degree of molecular orientation of polymer chains along a direction of applied stress. Molecular orientation refers to the relative orientation of polymer chains along a longitudinal or covalent axis of the polymer chains. A polymer with a high degree of molecular orientation has polymer chains that are aligned or close to being aligned along their covalent axes.

Polymers in the solid state may have amorphous regions and crystalline regions. Crystalline regions include highly oriented polymer chains in an ordered structure. An oriented crystalline structure tends to have high strength and high modulus (low elongation with applied stress) along an axis of alignment of polymer chains.

On the other hand, amorphous polymer regions include relatively disordered polymer chains that may or may not be oriented in a particular direction. However, a high degree of molecular orientation may be induced by applied stress even in an amorphous region. Inducing orientation in an amorphous region also tends to increase strength and modulus along an axis of alignment of polymer chains. Additionally, for some polymers under some conditions, induced alignment in an amorphous polymer may be accompanied by crystallization of the amorphous polymer into an ordered structure. This is referred to as strain-induced crystallization.

Rearrangement of polymer chains may take place when a polymer is stressed in an elastic region and in a plastic region of the polymer material. A polymer stressed beyond its elastic limit to a plastic region generally retains its stressed configuration and corresponding induced polymer chain alignment when stress is removed. The polymer chains may become oriented in the direction of the applied stress which results in an oriented structure. Thus, induced orientation in portions of a stent may result in a permanent increase in strength and modulus in that portion. This is particularly advantageous since after expansion in a lumen, it is generally desirable for a stent to remain rigid and maintain its expanded shape so that it may continue to hold open the lumen.

Therefore, radial expansion of a stent may result in deformation of localized portions. The deformation of the localized portions may induce a high degree of molecular orientation and possibly crystallization in the localized portions in the direction of the stress. Thus, the strength and modulus in such localized portions may be increased. The increase in strength of localized portions may increase the radial strength and rigidity of the stent as a whole. The amount of increase in radial strength of a stent may depend upon the orientation of the stress in the localized portions relative to the circumferential direction. If the deformation is aligned circumferentially, for example, the radial strength of the expanded stent can be increased due to the induced orientation and possibly strain induced crystallization of the localized portions. Thus, plastic deformation of localized portions may cause the portions to be “locked” in the deformed state.

Furthermore, induced orientation and crystallization of a portion of a stent may increase a T.sub.g of at least a deformed portion. The T.sub.g of the polymer in the device may be increased to above body temperature. Therefore, barriers to polymer chain mobility below T.sub.g inhibit or prevent loss of induced orientation and crystallization. Thus, a deformed portion may have a high creep resistance and may more effectively resist radial compressive forces and retain the expanded shape during a desired time period.

As depicted in FIGS. 3A and 3B, the second radially expandable undulating cylindrical rings 201 are longitudinally aligned and across the middle of each beecomb, cells to form circumferentially multiple X-shaped patterns. As pairs 201 of radially expandable undulating cylindrical rings is significantly shorter than that of first pair of pairs radially expandable undulating cylindrical rings 101, each strut arm in this second radially expandable undulating cylindrical ring 201 are first being oriented during explanation and are therefore stiffer than the long-arm in the first undulating ring.

As indicated above, expansion of a stent tends to result in substantial deformation in localized portions of the stent pattern. Such deformation can result in induced polymer chain alignment and possibly strain induced crystallization, which may tend to increase the strength and modulus of these portions. When a stent having a pattern such as those depicted in FIGS. 2B and 3B is expanded, the second undulating rings will be expanded first and the molecular in the short bar arm tend to oriented along the circumferential direction.

As depicted in FIGS. 2B and 3B, the short bar arms in the second undulating rings are shorter than the long bar arms in the first undulating rings. The short bar arms tend to plastically deform prior to the long bar arms upon expansion. As discussed, above, the smaller the mass of a bar arm, the more readily it deforms under an applied stress. As stent 100 is expanded, short bar arms may tend to circumferentially align and become plastically deformed along their length. Therefore, the shorter bar arms may become permanently deformed or locked and rigid and act to provide resistance against recoil and inward radial forces.

Long bar arms, however, may tend to have a lower degree of circumferential alignment. As a result, the deformation of the longer bar arms may be completely or substantially elastic. Thus, the longer bar arms tend to be relatively elastic and provide flexibility to the stent. As indicated above, such flexibility is desirable due to cyclic forces imposed on the stent. Such flexibility is important in preventing cracking of the stent.

FIG. 5 depicts another embodiment of a stent pattern. In FIG. 5, a portion of a stem pattern 200 is also shown in a flattened condition so that the pattern can be clearly viewed. When the flattened portion of stent pattern 200 is in a cylindrical configuration, it forms a radially expandable stent.

FIG. 5 depicts pair 201 of second undulating cylindrical rings located at the both ends of the stent and the radiopaque pockets located inside the ring. A portion of stent 200 in FIG. 5 is shown in greater detail in FIGS. 6A and 6B.

When a stent having a pattern such as those depicted in FIGS. 6A and 6B is expanded, the stem have two more undulating rings at both ends. The short arm at both ends will further increase the radial strength and stent stability once being expanded radially.

Polymer implant embodiments may be nearly undetectable due to lack of mass density or absence of signal. Therefore, such embodiments may incorporate a radio opaque marker, such a radio opaque dots. Such dots may be produced by applying radiopaque material in paste form into rivet-like depressions or receptacles in or on the scaffold strut elements. As shown, regular patterns of radiopaque dot deposits on the scaffold would advantageously aid in the ease of radiological detection of such implant location.

In one embodiment, the medical device can be modified to include a radio-opaque material for detecting its location after deployment or to ascertain the effects of long-term use (6 months or 2 years). There are different types of modifications available, such as e.g. diffuse or spot marking of the scaffold. Accordingly the radio-opaque materials can be incorporated directly in the initial plastic composition either as an admixture or covalently bound component. Alternatively, the radio-opaque material can be placed in a plurality of specific spot receptacles regularly distributed on or in the scaffold. Or the radio-opaque materials can be applied as part of a thin coating on the scaffold.

Therefore, the contrast detection enhancement of tissue implants by electron-dense or x-ray refractive markers are advantageous. Such markers can be found in biodegradable spot depots filled with radiopaque compositions prepared from materials known to refract x-radiation so as to become visible in photographic images. Suitable materials include without limit, 10-90% of radiopaque compounds or microparticles which can be embedded in biodegradable moieties, particularly in the form of paste like compositions deposited in a plurality of cup shaped receptacles located in preformed polymeric scaffold strut elements.

The radiopaque compounds can be selected from x-radiation dense or refractive compounds such as metal particles or salts. Suitable marker metals may include iron, gold, colloidal silver, zinc, and magnesium, either in pure form or as organic compounds. Other radiopaque material is tantalum, tungsten, platinum/iridium, or platinum. The radiopaque marker may be constituted with a binding agent of one or more aforementioned biodegradable polymer, such as PLLA, PDLA, PLGA, PEG, etc. To achieve proper blend of marker material a solvent system is includes two or more acetone, toluene, methylbenzene, DMSO, etc. In addition, the marker depot can be utilized for an anti-inflammatory drug selected from families such as PPAR agonists, steroids, mTOR inhibitors, Calcineurin inhibitors, etc. In one embodiment comprising a radioopaque marker, irons containing compounds or iron encapsulating particles are cross-linked with a PLA polymer matrix to produce a pasty substance which can be injected or otherwise deposited in the suitably hollow receptacle contained in the polymeric strut element. Such cup-like receptacles are dimensioned to within the width of a scaffold strut element. Heavy metal and heavy earth elements are useful in variety of compounds such as ferrous salts, organic iodine substances, bismuth or barium salts, etc. Further embodiments can utilize natural encapsulated iron particles such as ferritin that may be further cross-linked by cross-linking agents. Furthermore, ferritin gel can be constituted by cross-linking with low concentrations (0.1-2%) of glutaraldehyde. The radioopaque marker may be applied and held in association with the polymer in a number of manners. For example, the fluid or paste mixture of the marker may be filled in a syringe and slowly injected into a preformed cavity or cup-like depression in a biodegradable stent strut through as needle tip. The solvents contained in the fluid mixture can bond the marker material to the cavity walls. The stent containing radiopaque marker dots can be dried under heat/vacuo. After implantation, the biodegradable binding agent can breakdown to simple molecules which are absorbed/discharged by the body. Thus the radiopaque material will become dispersed in a region near where first implanted.

The stent patterns disclosed herein are not limited in application to stents. The pattern may also be applied to other implantable medical devices including, but not limited to, self-expandable stents, balloon-expandable stents, stent-grafts, and vascular grafts.

Stent patterns for polymeric stents may be formed from a polymeric tube by laser cutting the pattern of struts in the tube. The stent may also be formed by laser cutting a polymeric sheet, rolling the pattern into the shape of the cylindrical stent, and providing a longitudinal weld to form the stent. Other methods of forming stems are well known and include chemically etching a polymeric sheet and rolling and then welding it to form the stent.

Polymer tubes used for fabricating stents may be formed by various methods. These include, but are not limited to, extrusion and injection molding. A tube used for fabricating a stent may be cylindrical or substantially cylindrical in shape. Conventionally extruded tubes tend to possess no or substantially no radial orientation or, equivalently, polymer chain alignment in the circumferential direction. In some embodiments, the diameter of the polymer tube prior to fabrication of an implantable medical device may be between about 0.2 mm and about 5.0 mm, or more narrowly between about 1 mm and about 3 mm.

Representative examples of polymers that may be used to fabricate embodiments of implantable medical devices disclosed herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyp hosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxyethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

In one embodiment, pharmaceutical compositions can be incorporate with the polymers by, for example, admixing the composition with the polymers prior to extruding the device, or grafting the compositions onto the polymer active sites, or coating the composition onto the device.

### EXAMPLES

An embodiment of the present invention is illustrated by the following set forth example. All parameters and data are not to be construed to unduly limit the scope of the embodiments of the invention.

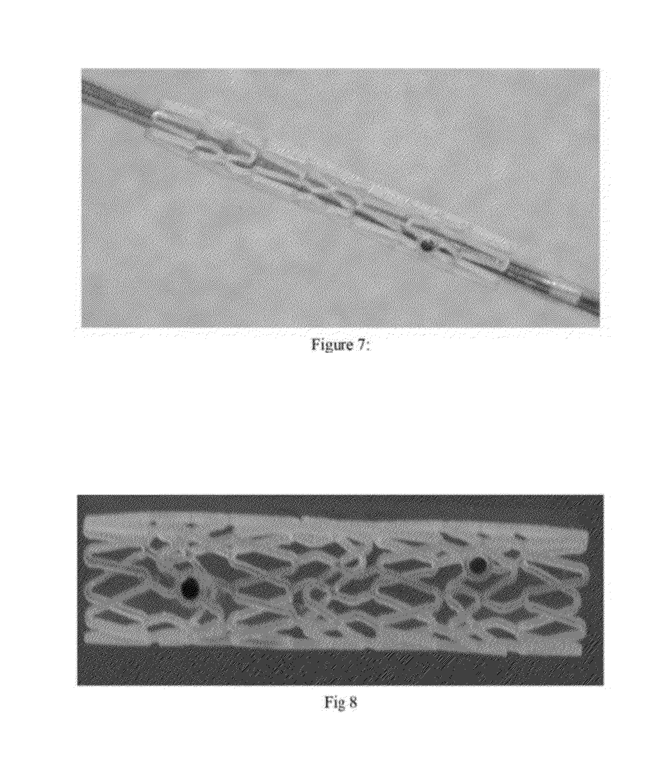

FIG. 7 depicts an invented biodegradable stent crimped on a balloon catheter. As depicted in the figure, the crimped biodegradable stent has a minimum acceptable profile.

FIG. 8 depicts the biodegradable stent in an expanded condition. As depicted in the figure, metal makers were located inside the strut.

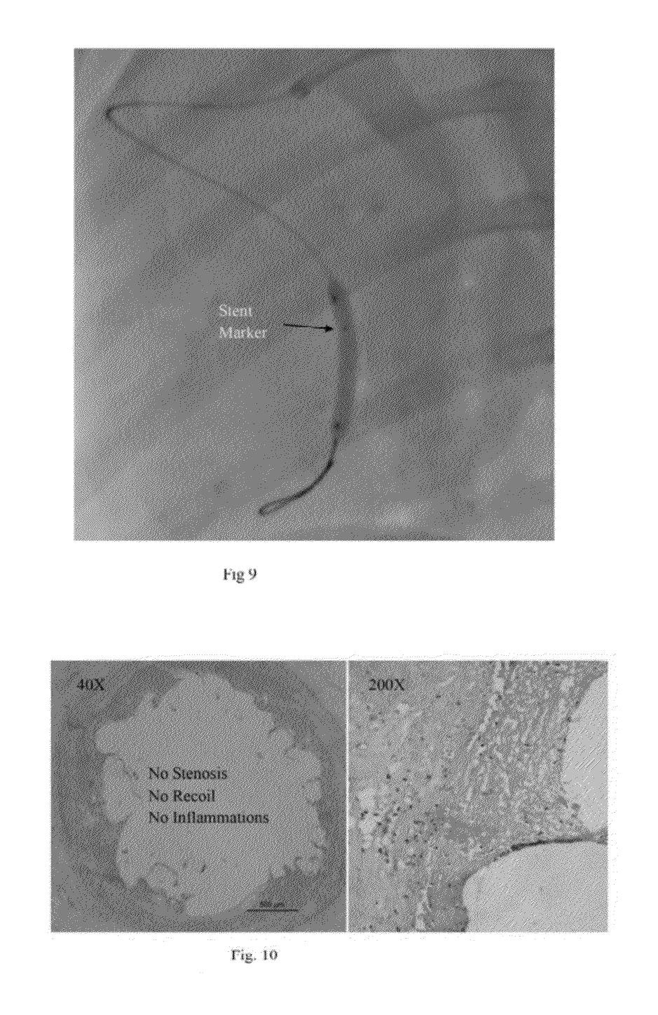

FIG. 9 depicts an angiography of described biodegradable stent in pig coronary artery at implantation. As depicted in figure, the biodegradable stent is radiolucent, but radiopaque marker is clearly identified.

FIG. 10 depicts the pathological images of invented biodegradable stent at one month post implantation in pig coronary artery. As depicted, there are no any indication of stent recoil, restenosis formation and arterial tissue inflammation at one month post implantation.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An expandable tube-shaped scaffold having a proximal end and a distal end defined about a longitudinal axis, said scaffold comprising: a) the first plurality of pairs of radially expandable undulating cylindrical rings that are longitudinally aligned and are connected at a plurality of intersections by S-shaped links to form a plurality of beecomb-shaped cells. Each adjacent S-shaped links were sited in an opposite direction to provide adequate free space for the second plurality of ring to cross. b) a plurality of second radially expandable undulating cylindrical rings that have a shorter strut arm than the first radially expandable undulating cylindrical rings are longitudinally aligned across the middle of each beecomb-shaped cells to form circumferentially a series X-shaped patterns. c) The meandering among the first plurality of pairs of radially expandable undulating cylindrical rings, beecomb-shaped cells and series X-shaped second undulations along the longitudinal axis form a unique pattern that provides the device both the flexibility and radial strength once it being expanded.

2. The tube-shaped scaffold of claim 1, wherein the first plurality of pairs of radially expandable undulating cylindrical rings comprise: a plurality of pairs of radially expandable undulating cylindrical rings that are longitudinally aligned and are connected at a plurality of intersections to form a plurality of beecomb-shaped cells, each ring having a first delivery diameter and a second implanted diameter, wherein the ring comprises multiple v-shaped undulations with peaks located circumferentially between two intersections.

3. The tube-shaped scaffold of claim 2, wherein the total number of V-shaped undulations in the first plurality of pairs of radially expandable undulating cylindrical rings are greater than that in the second plurality of pairs of radially expandable undulating cylindrical rings, preferably, is double, more preferably is triple to that in second radially expandable undulating cylindrical rings.

4. The tube-shaped scaffold of claim 2, wherein the hoop perimeter of the first plurality of pairs of radially expandable undulating cylindrical rings at expanded configuration is longer than that in the second plurality of pairs of radially expandable undulating cylindrical rings, preferably, is double, more preferable is triple to that of in the second radially expandable undulating cylindrical rings.

5. The tube-shaped scaffold of claim 1, wherein the first plurality of pairs of radially expandable undulating cylindrical rings are longitudinally aligned and are connected at a plurality of intersections by S-shaped links to form a plurality of beecomb-shaped cells. Each ring having a first delivery diameter and a second implanted diameter.

6. The tube-shaped scaffold of claim 5, wherein the S-shaped linking structure is at the opposite direction, wherein an enlarged space among each beecomb-shaped cell was created to incorporate the second radially expandable undulating cylindrical rings crossing through.

7. The tube-shaped scaffold of claim 1, wherein the a plurality of second radially expandable undulating cylindrical rings comprise a plurality of pairs of radially expandable undulating cylindrical rings that are longitudinally aligned and across the middle of each beecomb-shaped cells to form circumferentially a series of X-shaped patterns. Each ring having a first delivery diameter and a second implanted diameter, wherein the ring comprises multiple V-shaped undulations with peaks located circumferentially between the valleys of the V-shaped undulation in the first plurality of radially expandable undulating cylindrical rings.

8. The tube-shaped scaffold of claim 7, wherein the total number of V-shaped undulation in the second plurality of radially expandable undulating cylindrical rings are lower than that in the first plurality of radially expandable undulating cylindrical rings, preferably, is twice, more preferably is three-time less than that in first plurality of radially expandable undulating cylindrical rings.

9. The tube-shaped scaffold of claim 7, wherein the hoop perimeter of the second plurality of pairs of radially expandable undulating cylindrical rings at expanded configuration is shorter that at in the first plurality of pairs of radially expandable undulating cylindrical rings, preferably, is twice, more preferable is three-time less than that in the second radially expandable undulating cylindrical rings.

10. The tube-shaped scaffold of claim 1, wherein the first plurality of pairs of radially expandable undulating cylindrical rings and second plurality of pairs of radially expandable undulating cylindrical rings are meandered to form a sinusoid pattern along the longitudinal axis.

11. The tube-shaped scaffold of claim 10, wherein the meandered sinusoid pattern comprise: a pair of first plurality of pairs of radially expandable undulating cylindrical rings with a second of plurality radially expandable undulating cylindrical rings in between, or a pair of plurality second radially expandable undulating cylindrical rings with a first plurality radially expandable undulating cylindrical rings in between.

12. The tube-shaped scaffold of claim 10, wherein the hoop perimeter of the second plurality of radially expandable undulating cylindrical rings at expanded configuration is shorter that at in the first plurality of radially expandable undulating cylindrical rings, preferably, is twice, more preferable is three-time less than that in the second radially expandable undulating cylindrical rings.

13. The tube-shaped scaffold of claim 10, wherein the total number of V-shaped undulation in the second plurality of radially expandable undulating cylindrical ring are lower than that in the first plurality of radially expandable undulating cylindrical rings, preferably, is twice, more preferably is three-time less than that in first plurality of radially expandable undulating cylindrical rings.

14. The tube-shaped scaffold of claim 10, wherein the meandered sinusoid pattern comprise a plurality of second radially expandable undulating cylindrical rings comprise a plurality of pairs of radially expandable undulating cylindrical rings that are longitudinally aligned and across the middle of each beecomb-shaped cells to form circumferentially a series of X-shaped patterns. Each ring having a first delivery diameter and a second implanted diameter, wherein the ring comprises multiple V-shaped undulations with peaks located circumferentially between the valleys of the V-shaped undulation in the first plurality of radially expandable undulating cylindrical rings.

15. The tube-shaped scaffold of claim 1, wherein said scaffold polymer undergoes a molecular reorientation and crystallization during the radial strain of expansion.

16. The stent of claim 15, wherein the second radially expandable undulating cylindrical rings are configured to plastically deform when the stent is expanded the second implanted diameter.

17. The tube-shaped scaffold of claim 1, wherein said scaffold comprises at least one attached or embedded identification marker.

18. The tube-shaped scaffold of claim 17, wherein said at least one attached or embedded identification marker comprises a spot radioopacity or a diffuse radioopacity.

19. The tube-shaped scaffold of claim 1 carried on an expandable balloon carrier device.

20. The tube-shaped scaffold of claim 1, wherein said scaffold comprises a polymer core material comprising at least one encapsulated drug for localized treatment of the vascular wall and lumen.

21. The tube-shaped scaffold of claim 20, wherein the at least one encapsulated drug is for the treatment and prevention of tissue inflammation and platelet aggregation.
